(19) United States
(12) Patent Application Publication
(10) Pub. No.: US 2003/0108600 A1
(43) Pub. Date: Jun. 12, 2003

(54) Ubidecarenone compositions and capsules containing the same

(75) Inventors: Fuminori Okibayashi, Shimizu-shi (JP); Takayuki Fukasawa, Shimizu-shi (JP)

Correspondence Address: OLIFF & BERRIDGE, PLC, P.O. BOX 19928, ALEXANDRIA, VA, 22320, US

(73) Assignee: SUN CAPSULE CO., LTD., Shimizu-shi (JP)

(21) Appl. No.: 10/222,925

(22) Filed: Aug. 19, 2002

(30) Foreign Application Priority Data
Aug. 21, 2001 (JP) 2001-250140

(51) Int. Cl. A61K 9/66; A61K 31/12; A61K 47/00

(52) U.S. Cl. 424/455; 514/690; 514/786

(57) ABSTRACT

To provide Ubidecarenone compositions being excellent in absorbability into the body, highly stable at low temperatures, keeping clear external appearance, and being liquid at ordinary temperature. Ubidecarenone is dissolved in oils by which the accumulation of fats in the body is low, for example, triglyceride, which contains a short chain fatty acid as constitutive fatty acid, preferably SALATRIM, or diglyceride, for example, a hybrid glyceride composed of a middle chain fatty acid and a long chain fatty acid.

### FIELD OF THE INVENTION

The present invention relates to highly absorbable Ubidecarenone compositions and capsules containing the compositions as the content.

### BACKGROUND ART

Ubidecarenone, namely, CoQ₁₀ or coenzyme Q10, having the following structural formula, has long been prescribed as an effective drug for treatment of the edema, pulmonary congestion, angina pectoris, and so on, caused by hypofunction of the heart. Recently, it has been approved as food and become a promising health material as a readily commercially available food diet for consumers who are much interested in their health.

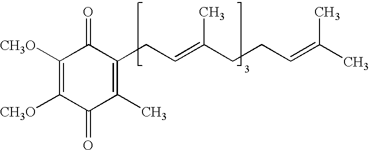

Ubidecarenone, however, is extremely poor in absorbability into the body and through the skin because of its very low solubility. Therefore, it has been proposed that Ubidecarenone is dissolved in edible natural and oils or middle chain fatty acid triglycerides, which are liquid at ordinary temperature, in order to improve absorbability in liquid forms (JP-A-54-92616(1979)).

Ubidecarenone dissolved in these solvents, however, is unstable at low temperatures to yield crystals of Ubidecarenone as precipitate during preservation, and therefore no improvement of absorbability is attained.

In order to improve the absorbability, it has also been proposed to use a hydrophilic surface-activating agent such as bile acid salts or HCO-60 in combination. In the use of such surface-activating agents, however, there is a possibility of causing disorders in gastric or gut mucosa (JP-B-64-10494(1989) corresponding to JP-A-56-18914(1981)).

In addition, emulsification (formation of cream) of Ubidecarenone compositions has been proposed, but no improvement of absorbability is recognized because of its insolubility. Moreover, emulsification is inconvenient for encapsulation since the sum of emulsion to the amount of Ubidecarenone to be added is increased excessively. Additionally, the appearance is not impressive in use as external materials for skin such as cosmetics because it turns unclear yellow.

Consumer's intention to purchase such goods as health food diets, quasi drugs, cosmetics, and soon is much influenced by his like or handiness, and therefore it is not possible to neglect their convenience and appearance. Since consumers who are much interested in health care choose, they would not like to orally ingest these goods containing natural oils that have an accumulative property of fats.

The so far proposed methods for liquefaction and emulsification make no great difference and are insufficient for providing the marketable goods not only in the field of drugs which require the pharmacological effect, but also in the field of health foods, quasi drugs or cosmetics.

### PROBLEMS THAT THE INVENTION IS TO SOLVE

The present invention was made in view of the above-mentioned so far unsolved problems and the purpose is to provide Ubidecarenone compositions characterized in that Ubidecarenone is dissolved to improve its absorbability.

In addition, the purpose of the invention is to provide widely applicable Ubidecarenone compositions being liquid at ordinary temperature, which are safe in oral digestion or skin permeation in human, contain the amount of Ubidecarenone as much as that making encapsulation possible, and have a clear and fine appearance, and further which can be recommended positively for use in the field of health foods, quasi drugs, cosmetics, and the like, as well as drugs, since solvents by which fats are scarcely accumulated in the body are employed.

Another purpose of the invention is to provide Ubidecarenone compositions of which the price is competitive with that of the goods purporting to contain the similar effective components.

### MEANS FOR SOLVING THE PROBLEMS

In order to solve the above-mentioned problems, the present inventor investigated a variety of dissolving liquids and found that Ubidecarenone is soluble in oils being liquid at ordinary temperature, particularly glycerides by which the accumulation of fats in the body is low, with no other defects as mentioned above. The following invention was completed based on the above-mentioned findings.

The invention of claim 1 provides a Ubidecarenone composition which is characterized in that Ubidecarenone is dissolved in a solvent comprising oils, being liquid at ordinary temperature, by which the accumulation of fats in the body is low.

The invention of claim 2 provides a Ubidecarenone composition as claimed in claim 1, wherein the solvent comprises a triglyceride having a short chain fatty acid as a constitutive fatty acid.

The invention of claim 3 provides a Ubidecarenone composition as claimed in claim 2, wherein the solvent comprises at least 60% of a triglyceride having a short chain fatty acid as a constitutive fatty acid.

The invention of claim 4 provides a Ubidecarenone composition as claimed in claim 2, wherein the triglyceride has a short chain fatty acid and a long chain fatty acid as constitutive fatty acids.

The invention of claim 5 provides a Ubidecarenone composition as claimed in claim 2, wherein the triglyceride has one or two short chain fatty acids and two or one long chain fatty acid as constitutive acids.

The invention of claim 6 provides a Ubidecarenone composition as claimed in claim 2, wherein ethanol is further present in the Ubidecarenone composition.

The invention of claim 7 provides a Ubidecarenone composition as claimed in claim 2, wherein ethanol is further present in an amount of 10 by weight or less to the Ubidecarenone composition.

The invention of claim 8 provides a capsule comprising a Ubidecarenone composition as the contents, the Ubidecarenone composition being dissolved in a solvent comprising a triglyceride having a short chain fatty acid as a constitutive fatty acid.

The invention of claim 9 provides a capsule as claimed in claim 8, wherein the capsule is a soft capsule.

The invention of claim 10 provides a Ubidecarenone composition as claimed in claim 1, wherein the solvent comprises a diglyceride.

The invention of claim 11 provides a Ubidecarenone composition as claimed in claim 10, wherein the solvent comprises at least 60% of a diglyceride.

The invention of claim 12 provides a capsule comprising a Ubidecarenone composition as the contents, the Ubidecarenone composition being dissolved in a solvent comprising a diglyceride.

The invention of claim 13 provides a capsule as claimed in claim 12, wherein the capsule is a soft capsule. a Ubidecarenone composition which is characterized in that Ubidecarenone is dissolved in a solvent comprising oils, being liquid at ordinary temperature, by which the accumulation of fats in the body is low.

### MODE FOR CARRYING OUT THE INVENTION

Hereinafter, the Ubidecarenone compositions relating to the mode for carrying out the invention will be explained according to the following items: 1. Fats and oils as solvents (drug solutions) for Ubidecarenone; 2. A process for producing Ubidecarenone compositions; and 3. Characteristics of Ubidecarenone compositions.

1. Solvents

The glycerides used as solvents for Ubidecarenone are liquid at ordinary temperature and include (A) triglycerides having (a) short chain fatty acid(s) as constitutive fatty acid, or (B) diglycerides. These are low calorie food oils.

(A) Triglycerides Having (a) Short Chain Fatty Acid(s) as Constitutive Fatty Acid

The triglycerides may be represented by the following structural formula:

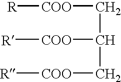

Wherein at least one of R—COO—, R′—COO— and R″—COO— is a short chain fatty acid. The short chain fatty acid is of 2-6 carbons, preferably of up to 4 carbons, including saturated or unsaturated and straight or branched chain. Acetic acid, propionic acid, normal butyric acid, iso-butyric acid, caproic acid, glycolic acid, lactic acid, hydroacrylic acid, hydroxybutyric acid, butenoic acid, pentanoic acid, hexanoic acid, and the like are exemplified.

The preferred triglycerides are constitutional lipids of low calorie vegetable oils constituting the above-mentioned short chain fatty acid(s) and (a) long chain fatty acid(s). In such a case, the long chain fatty acid includes saturated fatty acids of 16-40 carbons, preferably 16-24 carbons, more preferably 16-22 carbons. As for the long chain fatty acids, palmitic acid, stearic acid, arachidic acid, behenic acid, lignoceric acid, cerotic acid, montanic acid, melissic acid, and the like are exemplified. As sources of the long chain fatty acids, vegetable oils such as soybean hardened oil or rapeseed (corolla) oil are preferred.

The above-mentioned triglycerides may be prepared by the ester exchange reaction of the long chain fatty acid source with a triester of short chain fatty acid.

Commercially available SALATRIM (or structured liquids having one or two long chain fatty acids and two or one short chain fatty acid), which is a liquid oil at ordinary temperature having the same freezing point as that of milk fat, has been utilized in preparation of milk products such as ice cream or baked cake. SALATRIM has been supplied commercially at low costs since it can be produced in an industrially large scale.

When the triglyceride having (a) short chain fatty acid(s) as constitutive fatty acid, particularly SALATRIM, is used as solvent, it is appropriate to use ethanol as a co-agent in combination because the stability at low temperature is further increased. Specific amount of ethanol to be combined may be adjusted depending on the amount or Ubidecarenone and the stock condition of the Ubidecarenone composition, though ethanol is usually added in an amount of 10% by weight or less to the Ubidecarenone composition to give a sufficient effect.

(B) Diglycerides

The diglycerides may be represented by the following structural formula.

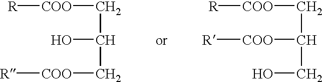

Wherein, the constitutive fatty acid (R—COO—, R′—COO—) is a middle chain fatty acid or a long chain fatty acid; the carbon number of the former is 6-11, and that of the latter 12-24. Preferably, the d glyceride is a hybrid composed of one middle chain fatty acid and one long chain fatty acid.

It has been reported that the diglyceride in which the fatty acid is located at the 1 and 3 positions, after digestion, is hydrolyzed in the duodenum to yield a 1-monoglyceride as a major product, and as a result, re-synthesis of triglycerides in the epithelial cells of the small intestine is inhibited to reduce accumulation of fats in the body or organs. Recently, it has also been reported that decrease of arteriosclerotic factors is effectively attained by setting the constitutive fatty acid in the range of (the amount of cis-type unsaturated fatty acid)/(the amount of saturated fatty acid+the amount of trans-type unsaturated fatty acid)≧6. Accordingly, it is recommended that the amount of the constitutive fatty acid is properly selected within the above-mentioned range.

The fatty acids may be saturated or unsaturated, and of straight or branched chain. Oleic acid, α-linolic acid, α-linolenic acid, dihomo-γ-linolenic acid, arachidonic acid, eicosapentaenoic acid, docosahexaenoic acid, palmitic acid, stearic acid, arachidic acid, and the like are exemplified.

The above-mentioned diglycerides may be prepared by the ester exchange reaction of a middle chain fatty acid source and a long chain fatty acid source with glycerin, followed by removal of by-product monoglycerides by molecular distillation or chromatography. The diglycerides may also be prepared by the ester exchange reaction of the triglyceride having a long chain fatty acid with a middle chain fatty acid in a condition under reduced pressure, followed by removal of by-product monoglycerides. They may also be synthesized by means of chemical syntheses.

The preferred long chain fatty acid source includes vegetable oils such as soybean hardened oil or rapeseed oil.

In terms of external appearance, the above-mentioned diglycerides are preferably clear liquids at room temperature.

The commercially available dissolving agent used in the invention is exemplified by an edible oil containing a diglyceride as major component, a product of Kao Corporation (trade name: ECONA). This edible oil has been supplied commercially at a low price as cooking oil for use at home. This commercially available good contains a small amount of by-products, i.e., monoglycerides and triglycerides, but no solubility of Ubidecarenone is spoiled.

2. A Process for Producing Ubidecarenone Compositions

Powdery Ubidecarenone is added to and mixed with a dissolving agent, and the mixture is stirred preferably under warming to dissolve sufficiently. The ratio of the combination (weight) is set approximately at Ubidecarenone/solvent=1: 8-15, in which ratio Ubidecarenone is dissolved enough.

3. Characterisitcs of Ubidecarenone Compositions

The Ubidecarenone compositions of the invention have good absorbability. The compositions are safe in oral digestion or skin permeation in human, exhibit excellent stability at low temperatures though the amount of solvent to be added is small, and give very beautiful impression since their appearance is yellowish and clear viscous liquid. Accordingly, encapsulation or keeping in transparent vessels is convenient regardless of soft capsule or hard capsule. The composition may make a good fill material of the capsules, especially the soft capsule. The capsules may be produced by conventional methods and apparatus. Of course, the composition may be encapsulated by conventional shell materials.

In a case of encapsulation, it is appropriate to consider formation of capsule coat containing a coloring agent such as titanium dioxide or caramel which is effective in shading the light in order to positively prevent oxidation.

### EXAMPLE

The following example will explain specifically that the Ubidecarenone compositions of the invention show marked effects.

(Preparation of Samples)

The following solvent was added to Ubidecarenone, and the mixture was stirred at 50° C. well for sufficient dissolution. When the dissolution was completed, the mixture was slowly cooled to room temperature to give a sample.

TABLE 1

| | Samp. 1 | Samp. 2 | Samp. 3 | Samp. 4 | Samp. 5 | Samp. 6 | Samp. 7 |
|---|---|---|---|---|---|---|---|
| Ubidecarenone | 10 | 10 | 10 | 10 | 10 | 10 | 10 |
| Solvent | | | | | | | |
| SALATRIM | 90 | — | — | — | — | — | 90 |
| Diglyceride | — | 90 | — | — | — | — | |
| Cholesterol | — | — | 90 | — | — | — | |
| Health ECONA | | | | | | | |
| Safflower oil | — | — | — | 400 | — | — | |
| Olive oil | — | — | — | — | 400 | — | |
| Middle chain | — | — | — | — | — | 300 | |
| fatty acid | | | | | | | |
| triglyceride | | | | | | | |
| Ethanol | — | — | — | — | — | — | 10 |

Among the above-mentioned solvents, SALATRIM is a product of DANISCO CULTOR A/S. Diglyceride is prepared as follows: a middle chain fatty acid of capric acid type is added to oleic acid monoglyceride, and allowed to react in the presence of an immobilized lipase catalyst at 60° C. for 6 hours under reduced pressure to yield a product consisting of 7% of monoglyceride, 75% of diglyceride and 12% of triglyceride, which product is purified by molecular distillaiton to give the aimed product containing 86% of diglyceride and 14% of triglyceride, of which the content of major fatty acids is 53% of oleic acid, 42% of caproic acid, 2% of stearic acid and 1% of linolic acid. Cholesterol health ECONA (trade name) is an edible oil containing a diglyceride as a major component (product of Kao Corporation).

The middle chain fatty acid triglyceride comprises glycerol binding to caprylic acid through an ester linkage.

Accordingly, Samples 1 to 3 and 7 are of the invention and Samples 4 to 6 are for comparison.

(Characterization)

Appearance

Samples 1 to 3 and 7 exhibited yellow and clear appearance.

Absorbability

Absorbability into the body can be examined by means of distribution of sodium cholate. 0.1% Sodium cholate aqueous solution and 0.5% sodium cholate aqueous solution, was placed respectively in 10 ml sample bottles, into which 2 or 3 drops of respective Samples were dropwise added, and the respective mixtures were vigorously stirred with a micro-spatula and allowed to stand for about 30 seconds to observe the state of the mixtures. The result was judged according to the following criteria.

OO . . . Yielding a light yellow homogeneous emulsion

O . . . A light yellow emulsion is yielded, but partially oily drops are floating on the surface of the water.

Δ . . . A part of Sample forms fine oil drops floating on the solution.

x . . . sample forms oil drops floating on the surface of the water.

TABLE 2

| | 0.1% Sodium cholate | 0.5% Sodium cholate |
|---|---|---|
| Sample 1 | OO | OO |
| Sample 2 | OO | OO |
| Sample 3 | OO | OO |
| Sample 4 | × | × |
| Sample 5 | × | × |
| Sample 6 | Δ | O |
| Sample 7 | OO | OO |

Stability at Ordinary Temperature and Low Temperature

Sample was placed in 10 ml sample bottles and preserved at room temperature or at 5° C. for a certain period. The state of crystallization was visually observed.

Texture

Texture is a factor for deciding the value of a good as cosmetics. Texture of Samples was examined in 5 monitor after the lapse of 20 days, and evaluated by them as follows: O: good; Δ: average; x; worse.

TABLE 3

| | Kept at room | Kept at room | Kept at | |
|---|---|---|---|---|
| | temperature | temperature | 5° C. | |
| | for 3 days | for 40 days | for 3 days | Texture |
| Sample 1 | No crystals | No crystals | No crystals | O |
| | precipitated | precipitated | precipitated | |
| Sample 2 | No crystals | No crystals | No crystals | O |
| | precipitated | precipitated | precipitated | |
| Sample 3 | No crystals | No crystals | No crystals | O |
| | precipitated | precipitated | precipitated | |
| Sample 4 | Crystals | Crystals | Crystals | × |
| | precipitated | precipitated | precipitated | |
| Sample 5 | Crystals | Crystals | Crystals | × |
| | precipitated | precipitated | precipitated | |
| Sample 6 | No crystals | Crystals | Crystals | × |
| | precipitated | precipitated | precipitated | |
| Sample 7 | No crystals | No crystals | No crystals | O |
| | precipitated | precipitated | precipitated | |

From the above-mentioned results, it was confirmed that the products of the invention are highly stable and much improved in absorbability in comparison with the reference standard, exhibit beautiful clear color, and have good texture.

Advantage of the Invention

As mentioned above, the Ubidecarenone compositions of the invention are greatly improved in their absorbability into the body. In addition, Ubidecarenone is dissolved stably in a small amount of the solvent to give a beautiful yellow clear viscous solution, of which the appearance is very agreeable. Moreover, since oils by which the accumulation of fats in the body is low can be used as solvent, they are good for health. Further, since the utilizable solvent has been supplied commercially at a low price, the Ubidecarenone compositions of the invention can be produced at a relatively low cost.

The compositions, accordingly, would induce customers to purchase sufficiently when they are launched on the market as health foods, quasi drugs, or cosmetics as well as drugs.

What is claimed is:

1. A Ubidecarenone composition which is characterized in that Ubidecarenone is dissolved in a solvent comprising oils, being liquid at ordinary temperature, by which the accumulation of fats in the body is low.

2. A Ubidecarenone composition as claimed in claim 1, wherein the solvent comprises a triglyceride having a short chain fatty acid as a constitutive fatty acid.

3. A Ubidecarenone composition as claimed in claim 2, wherein the solvent comprises at least 60% of a triglyceride having a short chain fatty acid as a constitutive fatty acid.

4. A Ubidecarenone composition as claimed in claim 2, wherein the triglyceride has a short chain fatty acid and a long chain fatty acid as constitutive fatty acids.

5. A Ubidecarenone composition as claimed in claim 2, wherein the triglyceride has one or two short chain fatty acids and two or one long chain fatty acid as constitutive acids.

6. A Ubidecarenone composition as claimed in claim 2, wherein ethanol is further present in the Ubidecarenone composition.

7. A Ubidecarenone composition as claimed in claim 2, wherein ethanol is further present in an amount of 10 by weight or less to the Ubidecarenone composition.

8. A capsule comprising a Ubidecarenone composition as the contents, the Ubidecarenone composition being dissolved in a solvent comprising a triglyceride having a short chain fatty acid as a constitutive fatty acid.

9. A capsule as claimed in claim 8, wherein the capsule is a soft capsule.

10. A Ubidecarenone composition as claimed in claim 1, wherein the solvent comprises a diglyceride.

11. A Ubidecarenone composition as claimed in claim 10, wherein the solvent comprises at least 60% of a diglyceride.

12. A capsule comprising a Ubidecarenone composition as the contents, the Ubidecarenone composition being dissolved in a solvent comprising a diglyceride.

13. A capsule as claimed in claim 12, wherein the capsule is a soft capsule.
